(19) United States
(12) Patent Application Publication
(10) Pub. No.: US 2013/0172635 A1
(43) Pub. Date: Jul. 4, 2013

(54) METHOD FOR PRODUCING FLUID, COLOURLESS POLYOCTENAMER BY RING-OPENING, METATHETIC POLYMERIZATION OF CYCLOOCTENE

(71) Applicants: Peter Hannen, Herten (DE); Norbert Wilczok, Muelheim (DE); Manfred Thiery, Coesfeld (DE); Martin Roos, Haltern am See (DE)

(72) Inventors: Peter Hannen, Herten (DE); Norbert Wilczok, Muelheim (DE); Manfred Thiery, Coesfeld (DE); Martin Roos, Haltern am See (DE)

(73) Assignee: EVONIK DEGUSSA GmbH, Essen (DE)

(21) Appl. No.: 13/806,925

(22) PCT Filed: May 26, 2011
(86) PCT No.: PCT/EP11/58657

§ 371 Date: Mar. 14, 2013

(30) Foreign Application Priority Data
Jun. 25, 2010 (DE) 10 2010 030 510.3

(51) Int. Cl. C07C 2/02; C07C 13/02

(52) U.S. Cl. CPC C07C 2/02; C07C 13/02
USPC 585/17; 585/318

(57) ABSTRACT

A polyoctenamer is described which at room temperature is liquid, colourless and clear, and which is produced by ring-opening, metathesis polymerization of cyclooctene.

### INTRODUCTION

The ring-opening, metathesis polymerization (ROMP=Ring Opening Metathesis Polymerisation) of cycloalkenes is well known.^{1.,2.} This reaction is catalysed by a number of transition metals, and a co-catalyst is often used here, combining with the transition metal complex to form the actual catalytically active species. Co-catalysts especially suitable are organylaluminium and organyltin compounds.

Other catalyst systems are based on defined transition metal complexes. Among the best known compounds are complexes based on ruthenium.^{3.,4.} However, a disadvantage here is that these are expensive, and especially that removal from the reaction product is difficult. Residues of ruthenium often cause unacceptable coloration of the product. In these instances, the polymer requires purification by complicated processes, e.g. reprecipitation, and this is a hindrance to economic production.

An essential factor in the polymerization process is the possibility of adjusting the properties of the resultant polymer. An example of another factor here, alongside the usual parameters, such as temperature, concentration of monomer, catalyst concentration and reaction time, is control of molecular weight distribution by way of addition of regulators, the function of which is to terminate the growing chain. Since a statistical process is involved, the molecular weight has, at first approximation, a reciprocal relationship to the concentration of regulator. No account is taken here of any broadening of the molecular weight distribution as a consequence of secondary metathesis (chain transfer or “back-biting”). Although addition of regulators can influence the average molecular weight—here given as Mw—it cannot influence the breadth of the molecular weight distribution. As the reaction proceeds further, secondary metathesis occurs, where the active end of a growing chain does not form an adduct with another monomer molecule but instead with the double bond of an existing polymer chain. The result is chain transfer, with a resultant increase in the non-uniformity or polydispersity (expressed as (Mw/Mn)−1 or Mw/Mn).

Another observation as reaction progresses is that the cis/trans-ratio shifts in favour of the trans-configuration, an effect which can likewise be attributed to secondary metathesis.

There is therefore a need for precise control of a very wide variety of process parameters, in order to adjust particular properties in the polymer.

### Object:

The polymerization of cyclooctene by ROMP is an important process for producing Vestenamer®, a polyoctenamer with an average molar mass of >100 000 g/mol. However, some applications require that the polymer is liquid at room temperature. An important application for a polyoctenamer which at room temperature is liquid and also colourless is the barrier property in relation to oxygen, carbon dioxide, water, etc., in films used for packaging. In order to achieve these properties, the physical parameters given in Table 1 must be achieved.

TABLE 1
Physical properties (preferred ranges) of
the polyoctenamer according to the invention:

| | |
|---|---|
| M_{w} [g/mol] | from 15 000 to 20 000 |
| cis-content [%] | from 65 to 70 |
| Melting point [° C.] | from −10 to 10 |
| J value [mL/g] | from 40 to 50 |

Determination of viscosity value J in accordance with ISO 1628-1 at 23° C.:
Dissolve 10 g of Vestenamer in 11 of toluene
Schott Visco System AVS 500 measurement equipment
No. 53713 capillary from Schott

### Achievement of Object:

The object of producing a liquid and colourless polyoctenamer is achieved as described in the claims. The process according to the invention achieves the object in that the reaction is terminated prior to or on achievement of full conversion. This gives a cis-trans-ratio in the range given, resulting in lower crystallinity of the polymer, and the melting point can therefore be kept low. Optionally incomplete conversion does not imply any disadvantage, because problem-free removal and recycling of unreacted cycloalkene is possible. The reaction is terminated at from 30 to 100% conversion, preferably from 50 to 100%.

Another essential aspect of the process is restriction of molecular weight through use of regulators which, as mentioned in the introduction, restrict chain growth.

In particular, bulk polymerization has proved successful, alongside the reaction in solution, in the process according to the invention. The parameters that are of importance for the process according to the invention are described below.

### Catalyst System:

The preferred catalyst system is composed of a mixture of tungsten hexachloride (WCl₆) and ethylaluminium dichloride (EtAlCl₂). The ratio of EtAlCl₂ to WCl₆ is preferably from one to six. A ratio of from two to five is particularly preferred. It is possible to use acidic compounds, such as alcohols, to activate the precatalyst. Other suitable materials alongside ethylaluminium dichloride are ethylaluminium sesquichloride and mixtures of ethylaluminium dichloride with diethylaluminium chloride in various ratios.

Amounts used are as follows: Tungsten hexachloride >0.1-0.04 mol %, particularly preferably from 0.1 to 0.01 mol % (based on cycloalkene)

Ethylaluminium dichloride ->0.2-0.08 mol %, particularly preferably from 0.2 to 0.02 mol %. (based on cycloalkene)

### Solvent:

The monomer in the process according to the invention can be present in solution or in bulk. It is preferable to execute the reaction in hexane or toluene. Operations here are carried out at a concentration of from 20 to 60% by weight and particularly preferably at a concentration of from 40 to 60% by weight.

However, it is particularly preferable to carry out the polymerization without solvent.

### Temperature:

The process described can be operated isothermally or else adiabatically.

The temperature range is preferably from −20 to 120° C., depending on the monomers used and on the solvent. A particularly preferred temperature range is from 10 to 60° C. In the case of an adiabatic procedure, the temperature can be determined by way of parameters such as amount of catalyst, rate of addition, juncture of termination of the reaction, etc. The preferred temperature range here is from 20 to 50° C.

### Regulators:

As described, regulators which restrict molecular weight increase are added during the process according to the invention. These can by way of example involve acyclic alkenes having one or more non-conjugated double bonds which can be terminal or internal and which should bear no substituents. Examples of compounds of this type are pent-1-ene, hex-1-ene, kept-1-ene, oct-1-ene, pent-2-ene, etc. However, it is also possible to use cyclic compounds which have, in their side chain, a vinylic, allylic or higher-homologous double bond having a low degree of substitution, an example being vinylcyclohexene. Amounts used of vinylcyclohexene are from 2 to 7 mol %, particularly preferably from 3 to 6 mol % (based on starting material).

### Cycloalkenes:

The cycloalkenes used in the process according to the invention involve by way of example cyclobutene, cyclopentene, cycloheptene, cyclooctene, cyclodecene, cyclododecene, 1,5-dimethylocta-1,5-diene, 1,5,9-tetramethyldodeca-1,5,9-triene.

### Molar Mass:

The average molar masses achieved for the polyoctenamer according to the invention in the process described are from 10 000 to 50 000 g/mol.

Preference is given to an average molar mass of from 10 000 to 30 000 g/mol. Particular preference is given to an average molar mass of from 15 000 to 20 000 g/mol.

### Terminators:

Once the desired reaction time has been reached, the polymerization is terminated by deactivating the catalyst system. By way of example, a CH-acidic compound can be added for this purpose. Examples of compounds suitable for this purpose are alcohols, such as methanol, ethanol, propanol, etc., and also carboxylic acids, such as acetic acid.

### EXAMPLE

1.332 kg (12.09 mol) of cyclooctene are used as initial charge together with 0.065 kg (0.6 mol) of 4-vinylcyclohex-1-ene in a reactor under inert gas. 1.05 mL (18 mmol) of ethanol are added to a solution of 2.65 g (6.68 mmol) of tungsten hexachloride in 44 mL of toluene. The resultant precatalyst solution is added to the reactor, and 8 mL of a 20% solution of ethylaluminium dichloride in hexane are added dropwise by way of a cannula. Once a temperature of 35° C. has been achieved, the reaction is terminated by adding methanol. After all of the volatile constituents have been removed by distillation, a liquid polyoctenamer is obtained as product (yield 67% of theory). The properties of the resultant polyoctenamer are as follows: J value=56; DSC=−2.6° C. (corresponding to melting range); cis/trans=70/30.

J value, 23° C. in accordance with ISO 1628-1

DSC in accordance with ISO 11357 and DIN 53765

### REFERENCES

^{1.} ‘Olefin Metathesis and Metathesis Polymerization’, K. J. Ivin, J. C. Mol, Academic Press 1997.
^{2.} ‘Handbook of Metathesis’, Vol. 1-3, R. H. Grubbs, Wiley-VCH 2003.
^{3.} Weskamp, T.; Kohl, F. J.; Herrmann, W. A. J. Organomet. Chem. 1999, 582, 362-365. Weskamp, T.; Kohl, F. J.; Hieringer, W.; Gleich, D.; Herrmann, W. A. Angew. Chem. Int. Ed. 1999, 38, 2416-2419.
^{4.} Nguyen, S. T.; Johnson, L. W.; Grubbs, R. H.; Ziller, J. W. J. Am. Chem. Soc. 1992, 114, 3974-3975. Bielawski, C. W.; Grubbs, R. H. Angew. Chem. Int. Ed. 2000, 39, 2903-2906.

1. A polyoctenamer, which, at room temperature, is liquid, colourless, and clear.

2. The polyoctenamer of claim 1, having:
an average molar mass from 10 000 to 50 000 g/mol;
a cis-content from 65 to 70%;
a melting point from −10 to 10° C.; and
a J value in accordance with ISO 1628-1 at 23° C. from 40 to 50 ml/g.

3. A process for producing the polyoctenamer of claim 1, the process comprising
ring-opening, metathesis polymerizing cyclooctene in the presence of a catalyst and a regulator.

4. The process of claim 3, wherein the polymerizing is terminated at from 30 to 100% conversion of the cyclooctene.

5. The process of claim 3, wherein the catalyst comprises a mixture comprising tungsten hexachloride (WCl₆) and ethylaluminium dichloride (EtAlCl₂).

6. The process of claim 3, wherein the regulator comprises:
an acyclic alkene comprising a non-conjugated double bond, which can be terminal or internal and which bears no substituents; or
a cyclic compound comprising, in a side chain, a vinylic, allylic or higher-homologous double bond having a low degree of substitution.

7. The process of claim 3, wherein the polymerization is carried out in a solvent.

8. The process of claim 3, wherein the polymerization is carried out without solvent.

9. A packaging material comprising a film comprising the polyoctenamer of claim 1.

10. The process of claim 3, wherein the polymerizing is terminated at from 50 to 100% conversion of the cyclooctene.

11. The process of claim 5, wherein a content of the tungsten hexachloride is in a range from 0.01-0.1 mol % with respect to the cyclooctene.

12. The process of claim 5, wherein a content of the tungsten hexachloride is in a range from 0.04-0.1 mol % with respect to the cyclooctene.

13. The process of claim 5, wherein a content of the ethylaluminium dichloride is in a range from 0.02-0.2 mol % with respect to the cyclooctene.

14. The process of claim 5, wherein a content of the ethylaluminium dichloride is in a range from 0.08-0.2 mol % with respect to the cyclooctene.

15. The process of claim 13, wherein a content of the ethylaluminium dichloride is in a range from 0.08-0.2 mol % with respect to the cyclooctene.

16. The process of claim 7, wherein the solvent is hexane or toluene.

17. The process of claim 3, wherein the polymerizing is carried out at a temperature from −20 to 120° C.

18. The process of claim 3, wherein the polymerizing is carried out at a temperature from 10 to 60° C.

19. The polyoctenamer of claim 2, having an average molar mass from 10 000 to 30 000 g/mol.

20. The polyoctenamer of claim 2, having an average molar mass from 15 000 to 20 000 g/mol.
